(19) United States
(12) Patent Application Publication
(10) Pub. No.: US 2007/0099287 A1
(43) Pub. Date: May 3, 2007

(54) Cell picking tool comprising molded with cell-adhesive concave structure and cell handling method

(76) Inventors: Kay Teraoka, Aichi (JP); Takao Saito, Aichi (JP)

Correspondence Address: PATTERSON & SHERIDAN L.L.P., 595 SHREWSBURY AVE, STE 100, FIRST FLOOR, SHREWSBURY, NJ, 07702, US

(21) Appl. No.: 10/579,650

(22) PCT Filed: Nov. 19, 2004
(86) PCT No.: PCT/JP04/17266

§ 371 Date: Jul. 31, 2006

(30) Foreign Application Priority Data
Nov. 19, 2003 (JP) 2003-388637

(51) Int. Cl. C12M 3/00

(52) U.S. Cl. 435/287.1

(57) ABSTRACT

The present invention provides a cell picking tool, etc. The present invention relates to a molded body for cell collection (cell picking tool) with a concave structure capable of picking a cell sheet which has been two-dimensionally cultured in a culture container while keeping the sheet form without using a cell dispersant, wherein by placing the molded body for cell collection so as to come in contact with a cell assembly in a cell growth environment, a liquid component for cell growth (cell growth environment) is incorporated therein and cells can be collected in the concave structure while keeping the cells in a state of aggregation; and a method of handling the same.

### TECHNICAL FIELD

The present invention relates to a molded body with a concave structure capable of picking a cell sheet which has been two-dimensionally cultured in a culture container while keeping the sheet form without using a cell dispersant, and more particularly relates to a molded body for cell collection (cell picking tool) that incorporates a liquid component for cell growth (cell growth environment) and can collect cells in the concave structure while keeping the cells in a state of aggregation by placing the molded body for cell collection so as to come in contact with a cell assembly in a cell growth environment.

In a technical field in which a method of culturing biological cells and cultured cells produced by the method are utilized, the present invention is useful to provide a tool that enables a new cell handling method having unprecedented and excellent characteristics that cells cultured, for example, in a dish like a conventional method can be collected without requiring the step of releasing the cells with a cell releasing agent such as trypsin; cells can be seeded and subcultured in an arbitrary cell growth environment by transferring a molded body with which the cells have been collected; cells incorporated into the molded body can be formed into a two-dimensionally or three-dimensionally accumulated matter by a simple operation; different cells can be gathered and cultured at one site together with the molded bodies retaining the respective suitable growth environments; and the like.

The cell picking tool of the present invention provides a new cell handling technique in the field of cell research contributing, for example, to medical techniques and genome sciences, and can be suitably used in a new cell handling method or the like in, for example, less invasive collection and subculture of cells, three-dimensional cell culture, cell therapy or co-culture.

### BACKGROUND ART

Cell culture techniques accumulated as basic techniques that support biotechnology are powerful tools for handling cells in fields of, for example, tissue engineering, regeneration medicine and drug discovery. In these fields, it is demanded that cultured cells are in the form of sheet or aggregation so as to capable of handling the cells. A cell culture mode which is most generally performed at present is a two-dimensional culture on a culture container such as a culture dish or a plate. In a two-dimensional culture system, cells adhere to the bottom surface of a culture container, therefore it is advantageous for cell growth, however, the cultured cells are strongly restrained in the culture container. Due to this, when the cells cultured in the two-dimensional culture system are subcultured or handled, the step of releasing the cells from the culture container by a treatment with a cell dispersant such as a trypsin treatment is essential. However, according to the above-mentioned method, there is a problem that the cultured cells cannot avoid damage caused by the cell dispersant. Further, in the above-mentioned method, because an extracellular matrix (ECM) is lost, there is a problem that it is extremely difficult to collect the cultured cells in a useful form (a sheet form or a block form) and to maintain the cell differentiation ability thereof.

As a conventional method for solving the above problems, for example, there is a method of culturing suspended cells by attaching the cells to the surface of a microcarrier (for example, Non-patent document 1 and Patent document 1). However, in the above-mentioned method, the step of suspending cultured cells to form a suspension is inevitable, and the cells cannot avoid physical stimulation (collision with the container or handling), therefore many cells result in death. In addition, because the surface of the microcarrier is a convex surface, it is difficult to obtain cells in the form of a dense aggregate. As another method, for example, a method of culturing cells by encapsulating cells with a gel such as calcium alginate has been also reported (for example, Patent document 2). However, also in the above-mentioned method, the step of suspending cultured cells to form a suspension is inevitable, and many cells result in death during the complicated encapsulation operation going through several stages. Further, because the cells are completely covered with the gel capsule, sufficient gas exchange cannot be maintained, therefore it is difficult to perform long-term culture. Further, the handling is difficult due to the lack of strength of the gel capsule.

In recent years, particularly in the field of regeneration medicine, there has been a strong demand for a method of collecting cultured cells in a useful form (a sheet form or a block form). As a technique in response to the above-mentioned demand, a method of suspension culture of cells on a non-cell adhesive substrate or a method of aggregating cells that are naturally released from a weak cell adhesive substrate has been reported. Further, related to the above methods, a method in which cells are cultured on a thin layer of poly(N-isopropyl acrylamide) that is shrunk at a temperature of 32° C. or higher and the layer is heated when the cells become confluent, and then the cells in the form of sheet are collected has been reported (for example, Non-patent document 2). Further, a method of obtaining a dense aggregate of cells by repeating a procedure in which cells are pelletized by centrifugation and the pelletized cells are suspension cultured has been studied (for example, Non-patent document 3). However, the cell sheet and the aggregate of cells produced by the above-mentioned methods are very fragile and cannot tolerate the clinical applications requiring cell handling with, for example, forceps or the like.

Patent document 1: JP-A-59-67965

Patent document 2: JP-A-10-248557

Non-patent document 1: L. Ikonomou et al., BIOTECHNOLOGY PROGRESS 18 (6), pp. 1345-1355, November-December, 2002

Non-patent document 2: T. Okano et al., J. Biomed. Mater. Res., Vol. 27, pp. 1243-1251, 1993

Non-patent document 3: Y. Kato et al., Proc. Natl. Acad. Sci. U.S.A., 85, 9552, 1988

### DISCLOSURE OF THE INVENTION

Problems that the Invention is to Solve

Under such circumstances, in view of the above-mentioned prior arts, the present inventors have made intensive studies for investigating a new cell handling tool that can surely solve the above-mentioned problems of the prior arts and a new application form thereof and a product thereof from several points of view, and as a result, they found that a desired object can be achieved by using a molded body with a concave structure capable of picking a part or the whole of cultured cells, an aggregate of cells or a biological tissue together with a given cell growth environment, thus the present invention has been completed.

That is, an object of the present invention is to provide a molded body for cell collection (cell picking tool) with which cultured cells in an appropriate culture environment can be incorporated into the molded body together with the culture environment and the cultured cells can be transferred to an objective culture environment together with the molded body. Further, another object of the present invention is to provide a method of subculturing cells cultured in a culture container such as a dish in another culture environment using the above-mentioned cell picking tool. Further, another object of the present invention is to provide a method of co-culturing a plurality of cultured cells by transferring and placing them in a single objective culture environment together with the cell picking tools retaining the respective culture environments.

Further, another object of the present invention is to provide a method of forming an appropriate aggregate of cells, for example, by utilizing the concave structure of a molded body retaining a cell growth environment. Further, for example, in the case where the molded body is composed of a substance that is harmless to a living body, another object of the present invention is to provide an application of an aggregate of cells formed in the concave structure of the molded body as an injectable filler for a living body together with the molded body.

Means for Solving the Problems

The present invention for solving the above-mentioned problems is directed to a cell picking tool which is a molded body with a concave structure capable of picking a cell sheet which has been two-dimensionally cultured in a culture container while keeping the sheet form (while keeping the cells in a state of aggregation) without using a cell dispersant, characterized in that (a) the concave structure is made of a material having a cell adhesive property; (b) the area of the opening portion of the concave structure ranges from 100 to 9×10⁶ μm²; and (c) when the opening portion of the concave structure and the cell sheet are brought into contact with each other, the opening portion of the concave structure and the cell sheet adhere to each other. Preferred embodiments of this cell picking tool are as follows: (1) the molded body comprises a calcium phosphate ceramic; (2) the molded body is in a shape with a cross section having an aspect ratio (long axis/short axis) ranging from 1.005 to 5, and when it is placed on a flat surface, a part or the whole of an opening portion of a pore, a through hole or a dimple faces downward; (3) the molded body is a molded body mixture of one type or two or more types selected from the group consisting of a spherical shape, a bead shape, a block shape, a plate shape, a polyhedral shape, a chestnut bur-like shape, a dendritic shape and a protruding shape with a size ranging from 5×10⁻⁴ to 1×10³ mm³; and (4) the molded body has a structure of one type or two or more types selected from the group consisting of a pore, a through hole, a dimple, a slit, a joint formed by joining portions of protrusions, a surface adhesive protein, a hydrophilic-treated surface, a polymer coat and an oxide film.

Further, the present invention is directed to a molded body/cell complex in which cells are attached to the above-mentioned opening portion of the concave structure of the molded body.

Further, the present invention is directed to a two-dimensionally or three-dimensionally accumulated matter of the above-mentioned molded body/cell complexes.

Further, the present invention is directed to a cell handling method, characterized in that by placing the above-mentioned cell picking tool in a container (cell collection site) in which cells grow, cells are attached to the molded body side and allowed to grow (passive cell collection), whereby the cells are handled together with the cell picking tool.

Hereinafter, the present invention will be described in more detail.

In the present invention, the molded body is preferably made of, for example, a calcium phosphate ceramic (for example, hydroxyapatite, β-TCP or the like), or a single crystal thereof, polystyrene, collagen gel, gelatin, sodium alginate gel or an agar containing calcium phosphate at an appropriate concentration from the viewpoint of cell collection, however, it is not limited to these, and one having substantially the same effect or one similar to any of these can be used in the same manner. It is preferred that the area of the opening portion of the concave structure of the molded body ranges from 100 to 9×10⁶ μm² from the viewpoint of maintaining the structure of a cell sheet and satisfying both of the retention of a liquid component and cell infiltration. Further, it is preferred that the molded body is in the form of, for example, a spherical particle with a size ranging from about 5×10⁻⁴ to 1×10³ mm³ from the viewpoint of handling. Further, it is preferred that when the molded body is placed on a flat surface in a cell growth environment, a part or the whole of the opening portion of the concave structure of the molded body is made substantially orthogonal to the short axis of the molded body so as to bring a part or the whole of the opening portion of the concave structure of the molded body into contact with the cell growth flat surface.

The collection of cells with the use of the above-mentioned molded body is carried out by putting the molded body into a cell collection site in a culture dish or the like. The molded body is put into a cell collection site in a sterile state. As the sterilization method, for example, autoclave sterilization, gas sterilization, dry heat sterilization and UV sterilization can be exemplified, however, it is not limited to these and one having substantially the same effect or one similar to any of these can be used in the same manner. As described above, the cell collection site into which the molded body has been put is placed in an incubator which has been set so as to have an appropriate environment. In the above operation, the placing period may be arbitrary, however, it preferably ranges from 6 to 240 hours. The molded body which has been put into the cell collection site in a culture dish incorporates a liquid component for cell growth (cell growth environment) in the cell collection site, for example, serum, which is a medium component of the medium containing the serum on the surface or in the inside of the molded body and comes into contact with cells (FIG. 1). The cells that come into contact with the molded body adhere to the molded body using the surface of the molded body as a scaffold and are collected by the molded body (FIG. 2) (passive cell collection by the molded body). At this time, in the concave structure, in the case where the opening portion has a predetermined area, the cells are collected while keeping the cells in a state of aggregation. The cells collected by the micromolded body continue to grow and become confluent. In particular, in the concave structure of the micromolded body, the cells grow so as to fill the concave structure, resulting in forming an aggregate of cells (FIG. 3).

In the present invention, the cells are preferably collected from a cell assembly (cell collection site) in a culture container in which cells are cultured, for example, from arbitrary sub-confluent to confluent cells which have been two-dimensionally cultured in a culture dish, an aggregate of cells composed of 1×10⁴ cells or more in a culture dish, or the surface of a cell sheet formed in a suspension culture system. The above-mentioned culture containers are preferred in terms of cell growth. However, it is not limited to these, and one having substantially the same effect or one similar to any of these can be used in the same manner. In any of the above-mentioned cell collection sites, a medium that is suitable for the cells is contained. In the medium, for example, cells or the like are sometimes suspended at about 1×10⁶ cells/ml as needed. In the present invention, cells are collected from one or two or more cell collection sites selected from the above-mentioned sites.

The cells collected in the molded body are discharged to the outside of the molded body in a process of growth (FIG. 4). Further, at this time, when the molded bodies are adjacent to each other, the molded bodies are bridged by the growing cells, whereby a given structure can be maintained. That is, the molded body for cell collection (cell picking tool) according to the present invention shows preferably, for example, a cell adhesive property and is composed of a molded body with a concave structure that can satisfy both of the maintenance of the structure of a cell sheet and the retention of a liquid component. When the opening portion of the concave structure of the cell picking tool and the cell sheet are brought into contact with each other, a function of attaching the opening portion of the concave structure to the cell sheet is exerted. Therefore, by placing a sterilized cell picking tool in a cell collection site, the cell growth environment in the cell collection site and the cells can be infiltrated and grown in the cell picking tool. By collecting the cell picking tool in which cells are infiltrated and grown and transferring it to another culture environment, the cells collected in the cell picking tool are discharged to the place where the cell picking tool is transferred, whereby arbitrary cells can be seeded and subcultured in a desired culture environment. Further, by arranging arbitrary cells collected in the cell picking tool in a three dimensional manner together with the cell picking tool, an aggregate of cells with a three-dimensional cell culture system and a three-dimensional cell structure can be formed. Further, in the present invention, for example, in the case where the cell picking tool is composed of a substance that is harmless to a living body, the aggregate of cells formed in the concave structure of the cell picking tool can be formed into an injectable filler for a living body together with the cell picking tool. However, the present invention is not limited to these methods.

The present invention is applied to various uses by passively collecting cells cultured by an appropriate method, for example, a two-dimensional culture method using a culture dish that is advantageous for cell growth in a molded body thereby efficiently and effectively handling the cultured cells. According to a conventional method, cultured cells are once released from a culture container with a cell dispersant such as trypsin, thereby preparing a cell suspension, and then, the cell suspension is applied to various uses. However, the cultured cells collected with a cell dispersant lose almost all the extracellular matrix (ECM) which contributes to cell growth, differentiation and the like, therefore, they are not suitable for the application, for example, to cell therapy, tissue engineering or the like. On the other hand, according to the present invention, cultured cells can be collected in a molded body capable of a handling operation without using a cell dispersant. The method of the present invention in which a cell dispersant is not used is less invasive to cells, and the obtained cultured cells retain plenty of useful ECM. Further, according to the present invention, cells collected in the molded body continue to grow even in the molded body, therefore, a dense aggregate of cells that survive over a long period of time can be produced.

The cells collected by the molded body can be transferred together with the molded body. In particular, the cells collected in the through hole or the concave structure of the molded body are not damaged with handling due to such as forceps unlike the case of a conventional method. In addition, because the molded body retains a liquid component necessary for cell growth, the cells are not dried during the transfer. Therefore, according to the present invention, cultured cells can be collected in a useful form in terms of cell therapy, tissue engineering or the like. Further, by arranging the molded bodies with which cells have been collected in a desired three-dimensional structure, a three-dimensional cell culture system and three-dimensional structured cultured cells can be constructed.

With the full use of the cell picking tool of the present invention, for example, by transferring the molded body with which cultured cells have been collected to a new dish, subculturing and seeding of cells can be carried out less invasively. Further, different cells cultured in appropriate culture methods can be co-cultured by collecting the different cells with the cell picking tools of the present invention and transferring the cell picking tools to an arbitrary culture environment. Further, in the case where the cell picking tool is composed of a substance that is harmless to a living body, the cell picking tool with which cells constituting a tissue desired to be regenerated have been collected is surely placed on a region to be treated, whereby tissue regeneration can be supported (cell therapy) . For example, in the case where bone cells or cells that may be differentiated into bone are collected in a molded body made of calcium phosphate (such as hydroxyapatite), they can be formed into an injection for hard tissue regeneration.

The cell picking tool of the present invention will be a commercial product as a kit obtained by sterilizing and packing the tool. For example, a given product can be produced by packing the cell picking tool in the space of an appropriate bag or capsule thereby preparing a filler, and sterilizing and packing the filler, and then combining it with an appropriate cell culture environment. In this case, the cell picking tool can be formed into a filler by mixing it with an appropriate cell culture environment. Further, in the present invention, a filler can be formed by allowing the above-mentioned cell picking tool to carry a given pharmaceutical ingredient. As the given pharmaceutical ingredient, for example, an antibiotic, an anti-inflammatory agent, platelet-rich plasma, BMP and the like can be exemplified. However, it is not limited to these, and it is possible to allow the tool to carry an appropriate pharmaceutical ingredient.

Advantage of the Invention

The present invention relates to a new cell collection medium that enables a handling operation of cells together with a molded body using a molded body/cell complex obtained by placing the molded body at a cell assembly in a cell culture container in which cells grow (cell collection site), thereby incorporating a liquid component for cell growth (cell growth environment) in the molded body and allowing the cells to adhere to the molded body side and to grow (passive cell collection). According to the present invention, the following special operational advantages can be obtained: (1) cultured cells can be collected, transferred and seeded without using a cell dispersant such as trypsin; (2) cell collection using the above-mentioned cell collection medium is less invasive to cells; (3) subculture of cells can be simply carried out with the full use of the above-mentioned cell collection medium; (4) an aggregate of cells with a three-dimensional structure can be produced with the full use of the above-mentioned cell collection medium; (5) a plurality of cultured cells can be gathered and co-cultured at an arbitrary one site with the full use of the above-mentioned cell collection medium; and (6) intuitive handling of an aggregate of cells formed in the above-mentioned cell collection medium can be carried out, whereby it is useful as an injectable filler for a living body in response to regeneration medicine.

### BEST MODE FOR CARRYING OUT THE INVENTION

Hereinafter, the present invention will be specifically described with reference to Examples, however, the present invention is by no means limited to the following Examples.

### EXAMPLE 1

Hydroxyapatite (HA) powder prepared so as to have a particle size of 50 μm or less was mixed in 1 wt % aqueous sodium alginate solution to give a final concentration of 20 wt% and a uniform slurry was prepared. An HA gel ball with a diameter of 1.6 mm was formed by dropping the slurry into 1 wt % aqueous calcium chloride solution. By dropping the slurry using a digital pipette, the size of the apatite gel ball was controlled. Before the HA gel ball was dried, a through-hole passing through the center of the HA gel ball was made with a stainless wire with φ500 μm. By this operation, the HA gel ball could be flattened in the direction of the through-hole. The HA gel ball was dried at 60° C. for 12 hours after the through-hole was made, and then sintered at 1200° C. for 1 hour. Thus, an HA ceramic bead having a long axis of 1 mm and a short axis of 0.9 mm (aspect ratio: 1.11) and having the above-mentioned 300 μm-through-hole (cell picking tool, HAB) was produced. The produced HAB was stable in a state in which the opening portion of the through-hole was facing toward the bottom on a flat surface (FIG. 5).

### EXAMPLE 2

The HAB produced in Example 1 was subjected to ultrasonic cleaning in 99.5% ethanol for 10 minutes. Human osteosarcoma cells MG63 were cultured in 6 wells (9.6 cm² in diameter/well) of a culture dish and made in a sub-confluent state. In the above-mentioned culture, Dulbecco's MEM+10% FBS+1% P.S. was used as a medium. The HAB subjected to dry heat sterilization at 200° C. for 2 hours were put into the culture dish at 20 balls/well and placed in an incubator at 37° C. under 5% CO₂. By the above-mentioned operation, MG63 on the culture dish could be collected in the HAB (FIG. 6). The amount of MG63 colleted in the HAB increased in proportion to the incubation time and reached 537, 2970 and 4728 cells/HAB after 24, 72 and 120 hours, respectively (FIG. 7). The amount of MG63 colleted in the HAB molded body was calculated based on the amount of DNA extracted from the cells in the HAB. The viability (living cell ratio) of MG63 collected in HAB was 95% or higher. The HAB with which MG63 had been collected at 1-day incubation time was transferred to a 12-well culture dish and placed in an incubator with Dulbecco's MEM+10% FBS+1% P.S. as a medium. By the above-mentioned operation, MG63 could be seeded in the 12-well culture dish (FIG. 8). Thereafter, an aggregate of MG63 could be formed in the through-hole of the HAB.

### EXAMPLE 3

Sixty HAB molded bodies which had been produced in Example 2 and with which MG63 had been collected, were transferred to a 96-well culture dish, arranged three-dimensionally and placed in an incubator for 48 hours. As a result, the HAB molded bodies adjacent to each other could be bridged by the growing MG63 cells.

### EXAMPLE 4

HAB molded bodies with which a mouse osteoblast cell line MC3T3-E1 had been collected were produced in the same manner as in Example 2 except that the mouse osteoblast cell line MC3T3-E1 was used instead of MG63 in Example 2. The eight HAB molded bodies with which MC3T3-E1 had been collected and the eight HAB molded bodies which had been produced in Example 2 and with which MG63 had been collected were placed in an alternating fashion on a 12-well culture dish. As a result, a co-culture system in which MC3T3-E1 and MG63 were seeded in an alternating fashion could be produced.

### INDUSTRIAL APPLICABILITY

The present invention relates to a molded body with a concave structure that shows a cell adhesive property and can satisfy both of the maintenance of the structure of a cell sheet and the retention of a liquid component. According to the present invention, cells cultured in a cell collection site such as in a dish can be collected without releasing the cells with a cell dispersant, that is, while keeping the cells in a state of aggregation and can be seeded and subcultured in an arbitrary cell growth environment. The method of the present invention is less invasive to cells, and cultured cells can be handled without impairing useful ECM. Further, according to the present invention, by arranging molded bodies, cells incorporated into the molded bodies can be formed into a two-dimensionally or three-dimensionally accumulated matter. Further, according to the present invention, for example, different cells can be gathered and cultured at one site together with the molded bodies retaining the respective suitable growth environments. The cell handling system of the present invention is useful as a system that can realize less invasive collection and subculture of cells, three-dimensional cell culture, cell therapy, co-culturing method and the like. According to the present invention, a new tool for cell handling in fields of, for example, tissue engineering, regeneration medicine and drug discovery.

### BRIEF DESCRIPTION OF THE DRAWINGS

FIG. 1 shows a schematic diagram of a mode in which a molded body is put in a cell collection site in a culture dish, incorporates culture components including serum on the surface or in the inside thereof and comes into contact with cultured cells.

FIG. 2 shows a schematic diagram of a mode in which the cells that come into contact with the molded body adhere to the opening portion of the concave structure of the molded body and are collected while keeping the cell sheet structure (passive cell collection by the molded body).

FIG. 3 shows a schematic diagram of a mode in which the cells collected by the molded body continue to grow and become confluent, resulting in forming an aggregate of cells.

FIG. 4 shows a schematic diagram of a mode in which the cells collected in the molded body are discharged to the outside of the molded body.

FIG. 5 shows one example of a light microscope image of HA molded body (cell picking tool).

FIG. 6 shows one example of a light microscope image of MG63 on a culture dish collected by HA molded body.

FIG. 7 shows a graph showing the time course of the number of MG63 cells collected by HA molded body.

FIG. 8 shows one example of a light microscope image of MG63 cells seeded by HA molded body with which MG63 cells have been collected.

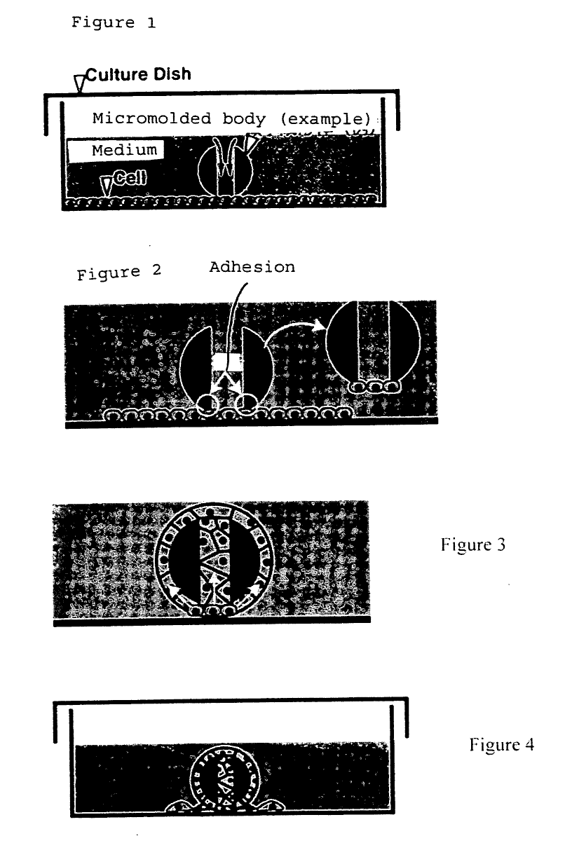

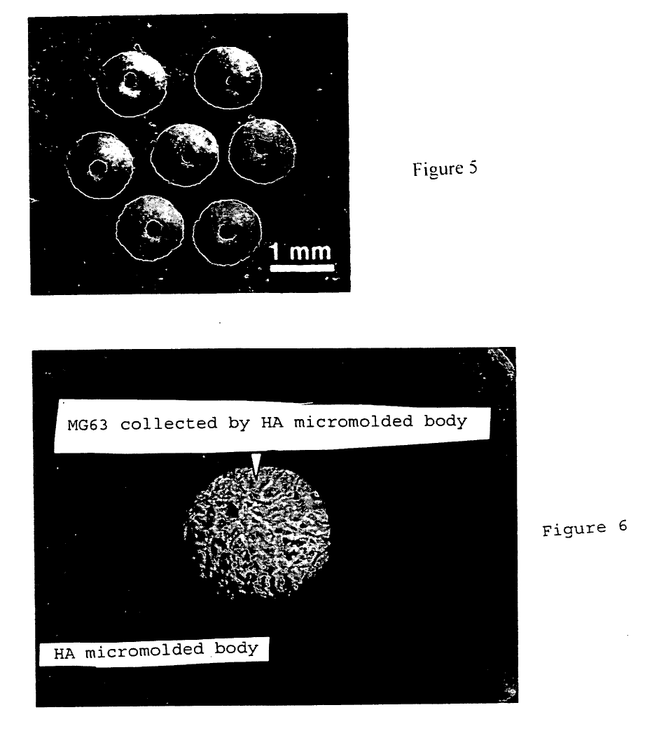

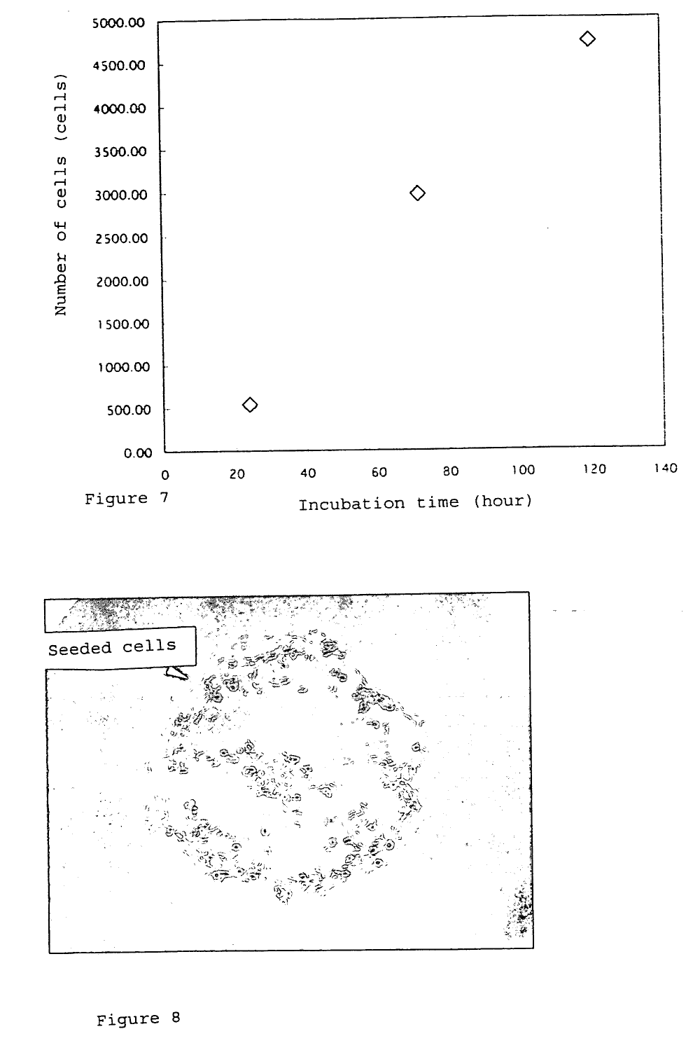

1. A cell picking tool which is a molded body with a concave structure capable of picking a cell sheet which has been two-dimensionally cultured in a culture container while keeping the sheet form (while keeping the cells in a state of aggregation) without using a cell dispersant, characterized in that:
(1) the concave structure is made of a material having a cell adhesive property;
(2) the area of the opening portion of the concave structure ranges from 100 to 9×10⁶ μm²; and
(3) when the opening portion of the concave structure and the cell sheet are brought into contact with each other, the opening portion of the concave structure and the cell sheet adhere to each other.

2. The cell picking tool according to claim 1, wherein the molded body comprises a calcium phosphate ceramic.

3. The cell picking tool according to claim 1, wherein the molded body is in a shape with a cross section having an aspect ratio (long axis/short axis) ranging from 1.005 to 5, and when it is placed on a flat surface, a part or the whole of an opening portion of a pore, a through hole or a dimple faces downward.

4. The cell picking tool according to claim 1, wherein the molded body is a molded body mixture of one type or two or more types selected from the group consisting of a spherical shape, a bead shape, a block shape, a plate shape, a polyhedral shape, a chestnut bur-like shape, a dendritic shape and a protruding shape with a size ranging from 5×10⁻⁴ to 1×10³ mm³.

5. The cell picking tool according to claim 1, wherein the molded body has one type or two or more types of structures selected from the group consisting of a pore, a through hole, a dimple, a slit, a joint formed by joining portions of protrusions, a surface adsorptive protein, a hydrophilic-treated surface, a polymer coat and an oxide film.

6. A molded body/cell complex in which cells are attached to the opening portion of the concave structure of the molded body according to claim 1.

7. A two-dimensionally or three-dimensionally accumulated matter of the molded body/cell complexes according to claim 6.

8. A cell handling method, characterized in that by placing the cell picking tool according to claim 1 in a container (cell collection site) in which cells grow, cells are attached to the molded body and allowed to grow (passive cell collection), whereby the cells are handled together with the cell picking tool.
